(19) United States
(12) Patent Application Publication
(10) Pub. No.: US 2005/0163649 A1
(43) Pub. Date: Jul. 28, 2005

(54) Candles with fragrance reservoirs and display cabinet

(76) Inventors: Eckhard K. Friedrich, Dallas, OR (US); Hans Damgaard, Ulfborg (DK)

Correspondence Address: Richard C. Litman;LITMAN LAW OFFICES, LTD., P.O. Box 15035, Arlington, VA, 22215, US

(21) Appl. No.: 10/762,317

(22) Filed: Jan. 23, 2004

(51) Int. Cl. A61L 9/00; A61L 2/08

(52) U.S. Cl. 422/1; 422/5; 422/26; 422/306

(57) ABSTRACT

A candle with a fragrance or scent reservoir comprising a wax candle incorporating a single or multiple reservoirs containing scented oil(s), scented wax stick(s), scented wax bead(s), or scented granules. The reservoir or reservoirs are formed in the candle body generally along one or more wicks. The reservoirs may also be formed in a wickless candle typically known as a tart or a melt candle. The scent is a perfume that can either be liquid, gel or solid. As the candle wick(s) burns and creates a melt pool, the fragrance materials mix with the melt pool and release the fragrances. A method of displaying and selling the candles uses a display cabinet with multiple shelves for displaying the various candles of various sizes, colors and shapes, and displaying different fragrance materials.

### BACKGROUND OF THE INVENTION

1. Field of the Invention

The present invention relates generally to candles and a display cabinet. More specifically, the invention is a candle having at least one scent material imbedded parallel to the wick. A display cabinet is designed to hold a variety of candles along with separate containers for scents in either individual bottles, tubes, concentrated wax sticks, pellets, grains or creams.

2. Description of the Related Art

The related art of interest describes various candles, but none discloses the present invention. There is a need for a candle with scents wherein a consumer can mix scents of one's choice and convenience to create from a variety of wax candles a candle that can release the selected scents when burning, and a suitable display cabinet with an assortment of shelves to feature the various perfumes and candles for sale for combination by the consumer.

The related art will be discussed in the order of perceived relevance to the present invention.

U.S. Patent No. U.S. 2002/0131909 A1 issued on Sep. 19, 2002, to Bernard I. Urfig describes a scented candle having three vertical shafts comprising a first centrally drilled hole approximately half-way from the top having a small diameter, a second hole having a large diameter drilled adjacent the center of the candle from the candle top but deeper, and a small diameter hole drilled from the bottom to the second hole. A wick is placed in the first centrally drilled hole. A heat conductive vial is inserted into the second hole, filled with a fragrance oil, and sleeved and capped until the candle is used. The candle is distinguishable for requiring a fragrance oil containing vial embedded in only in the top portion of the candle.

U.K. Patent Application Publication No. 2 229 734 A published on Oct. 3, 1990, for Phillip Y. T. Lam describes a scented candle comprising a core of wax, a wick extending through the wax core, an outer sealing layer of wax at least partially surrounding the core, and at least one layer of a scented composition located between the core layer and the outer layer such that as the wick and the candle are burned the scent diffuses into the atmosphere. The candle is distinguishable for requiring an intermediate vertical layer of a scented composition between the core and outer wax layers.

U.S. Pat. No. 6,544,303 B2 published on Apr. 8, 2003, for Jose F. Calzada describes a heat activated perfume pillar candle comprising a high load scented shell and a core candle with a similar or lesser fragrance load. The inside candle can be wax, paraffin, gel oil or polyamide-based, and can be scented or unscented. The outside shell can be paraffin wax and a reinforcement having a melting point of at least 70 degrees, and is scented to a level so that fragrance is continuously released without burning, and is further activated by lighting the core candle. The candle is distinguishable for requiring a high load scented shell and a core candle with a similar or less fragrance load.

U.S. Patent Application Publication published on Jun. 5, 2003, for Wendy M. Joyner describes a scented candle comprising an inner core having a higher concentration of fragrance that the outer shell can be hemispherical or square shaped. The wick is assumed to be centered and inserted after the wax body is formed. The candles need not be formed in glass jars or other containers. The candle is distinguishable for requiring a candle having a higher fragrance concentration in its outer shell.

U.S. Pat. No. 3,958,917 issued on May 25, 1976, to John F. Naz describes a scented ring for candles comprising a scented ring placed on top of the candle surrounding the wick. The candle is distinguishable for requiring a small wax ring impregnated by a fragrance.

U.S. Pat. No. 5,873,553 issued on Feb. 16, 1999, to Laura A. Spaulding describes fragrance-carrier compositions for use in tart candles comprising safe odorless base materials that have a “high throwing or dispersion power” for diffusing fragrances. The compositions contain hydrogenated polyolefins. The compositions are distinguishable for being limited to fragrance compositions for tart candles, and fail to describe specific structural additions.

U.S. Pat. No. 6,290,914 B1 issued on Sep. 18, 2001, to Gene M. LeJeune et al. describes a fragrance ring for oil lamps comprising an attachment having a rim portion to form a fragrance trough. The device is distinguishable for requiring an oil lamp.

U.S. Pat. No. 6,328,935 B1 issued on Dec. 11, 2001, to Felix Buccellato describes an aroma dispenser device for a candle comprising a candle for providing a flame, an open porous annular ceramic substrate adapted for absorbing and reversibly retaining the aromatic fluid, a support structure for holding the porous substrate in a coaxial spaced-apart relationship above the flame. The device is distinguishable for requiring a ceramic substrate impregnated with an aromatic fluid.

U.S. Pat. No. 6,426,051 B1 issued on Jul. 30, 2002, to Gerald H. Allison describes an oil-burning lamp adapted to disperse a fragrance comprising a lamp vase having vent holes proximate the open top portion. The device is distinguishable for requiring a lamp vase.

U.S. Pat. No. 6,435,694 B1 issued on Aug. 20, 2002, to Jeffrey Bell et al. describes a scentless candle housed in a vase having an outer portion and an inner portion recessed in the outer portion. The inner candle portion contains a wick. The outer portion may contain decorative effects. The candle device is distinguishable for requiring two distinct candle portions without any added scent.

Japan Patent Application No. JP2001-327588 published on Nov. 27, 2001, for Akira Yano describes an aroma generating vase device that allows various kinds of perfumes and aromas to exhibit the characteristics of the fragrances that these materials possess in their own form as they are. A cylindrical canopy having a through-hole is placed over a candle. Two in-line filters are placed in the through-hole and granular aroma generating agent is placed on top of the upper filter. A candle on the bottom of the device heats the aroma substance. The device is distinguishable for requiring a vase and aromatic impregnated filters.

PCT Patent Application No. WO 02/100449 A1 published on Dec. 12, 2002, for Deno Roumpos describes a candle warmer device comprising an open container having a scented candle on a heating device. The device is distinguishable for requiring a heater for heating a scented candle.

European Patent Application No. EP 1 310 264 A1 published on May 14, 2003, for John N. Wesley describes a fragrance dispensing device for an oil candle comprising a base receptacle containing a volatile fuel and a polymeric element containing a fragrance in another smaller receptacle on top having a wick extending from the fuel to the top of the first receptacle. The device is distinguishable for requiring a significantly different structure.

None of the above inventions and patents, taken either singly or in combination, is seen to describe the instant invention as claimed. Thus, various candles with one or more fragrance reservoirs and a display cabinet containing various candles and separate fragrance options solving the aforementioned problems is desired.

### SUMMARY OF THE INVENTION

The wax candles of various shapes and sizes have at least one and possibly several fragrance reservoirs for receiving a liquid or solid scenting material containing a fragrance. Multiple fragrances can be mixed in one candle, either by mixing the scenting material within a single reservoir, or by filling different reservoirs with differently scented materials. The selected scents in the form of liquids or solids are placed in vertical reservoirs parallel to the wick(s). Each reservoir may optionally be plugged to retain liquid scenting materials, such as scented oils, within the reservoir. As the candlewick burns and creates a melt pool, the scenting materials mix with the melted candle wax and release their fragrances.

A display cabinet has shelves for displaying the various candles and an additional area for the display of different scents. One curved shelf extends out for use by the consumer in selecting the desired candle and scents.

The candles can vary in cross-sectional shape such as round, square, rectangular, hexagonal, and octagonal, and in size such as 2 inch to 12 inches in diameter and from 3 inches to a foot in height. Furthermore, various finishing coats, colors and geometrical shapes such as floral and animals would be available.

The candles can be presented in jars of various sizes. For example, common jar candle sizes are 5 to 9 oz. (volumetric) for a small jar, 16 to 20 oz. for a medium jar, and 26 to 34 oz. for a large jar. Votive candles will be offered in standard votive sizes such as small, medium and large. Tea light candles, which are smaller versions of votive candles, are contained in smaller metal holders with one or more “scent wells”. The candles can have one or more wicks as mentioned above.

Tart, or melt, candles are wickless candles often used in a device known as a “tart burner”, or “candle warmer” where they are heated by electrical means such as a small heater, light bulb, or a stove top, or by an open flame such as a tea candle. When heated, scented tart candles release their fragrance. Scent reservoirs can be added to the tart candles in similar fashion to wicked candles, releasing a fragrance from the scenting material contained in the scent reservoirs when warmed.

The number of fragrances is numerous. A partial list of fragrances includes spice fragrances such as vanilla, allspice, almond, nutmeg, cinnamon, and clove, fruit fragrances such as strawberry, lime, pineapple, banana, apple, peach, cranberry, orange, raspberry, and pumpkin, floral fragrances such as gardenia, rose, violet, and lavender, and others such as pine, coffee, cider, and peppermint. Various combinations of the above mentioned fragrances are contemplated and selectable by the consumer.

At the present, all scented candles have the scent mixed with the wax at the factory level and require a retailer to use multiple display cabinets to provide an equal number of fragrance choices as presented in one cabinet in the present invention.

Accordingly, it is a principal object of the invention to provide both wicked and wickless candles having at least one reservoir for receiving and containing a scenting material.

It is another object of the invention to provide a candle having at least one reservoir for receiving and containing a scented oil.

It is yet another object of the invention to provide a candle having at least one reservoir for receiving and containing a stick of scented wax.

It is a further object of the invention to provide a candle having at least one plugged reservoir for receiving and containing a scented oil.

Still another object of the invention is to provide a display cabinet for displaying the candles along with the numerous separate fragrances to allow consumers to select and combine according to their desires.

Yet another object of the invention is to provide a variety of candles of various shapes and sizes displayed in a display cabinet having an extended shelf displaying numerous fragrances.

It is an object of the invention to provide improved elements and arrangements thereof for the purposes described which is inexpensive, dependable and fully effective in accomplishing its intended purposes.

These and other objects of the present invention will become readily apparent upon further review of the following specification and drawings.

### BRIEF DESCRIPTION OF THE DRAWINGS

FIG. 1 is an elevational side view of a candle with at least two fragrance reservoirs (one plugged) before lighting according to a first embodiment of the present invention.

FIG. 2 is an elevational side view of the candle shown in FIG. 1 as it is burning and releasing its two fragrances according to the present invention.

FIG. 3 is a top plan view of a first alternative embodiment of a candle having a single wick surrounded by three reservoirs for different fragrances.

FIG. 4 is top plan view of a second alternative embodiment of a candle having six peripheral fragrance reservoirs surrounding an inner circle of six wicks and a centered fragrance reservoir.

FIG. 5A is top view of a third alternative embodiment of a wickless tart, or melt, candle having three fragrance reservoirs.

FIG. 5B is side view of the wickless tart, or melt, candle shown in FIG. 5A.

FIG. 6A is a front elevational view of a specialized candle display cabinet having shelves for displaying candles along with scenting materials and having an extended removable shelf for selecting a consumer's fragrance selections according to a second embodiment of the present invention.

FIG. 6B is a side elevational view of the display cabinet of FIG. 6A cabinet illustrating the removable extended shelf.

FIG. 6C is a front perspective view of only the extended and removable sampling shelf of FIGS. 6A and 6B holding selected candles.

Similar reference characters denote corresponding features consistently throughout the attached drawings.

### DETAILED DESCRIPTION OF THE PREFERRED EMBODIMENTS

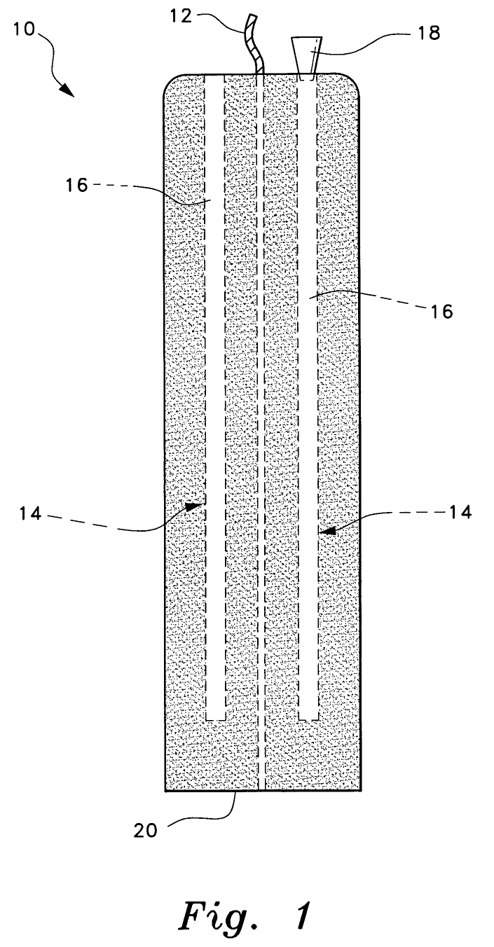

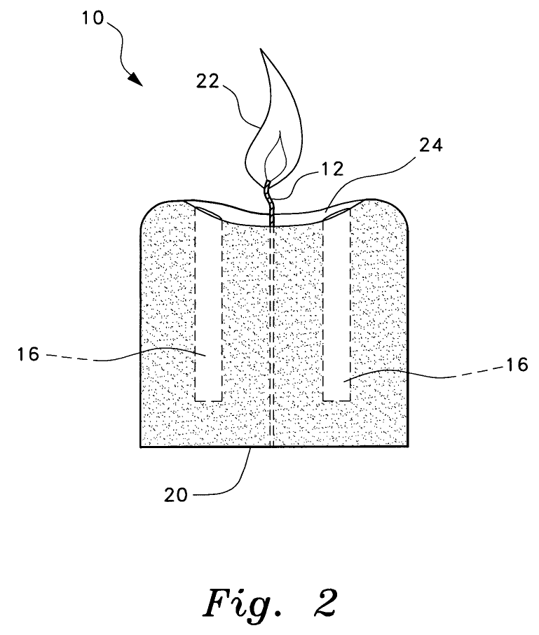

The present invention is directed in FIGS. 1 and 2 to a first embodiment of a fragrance containing candle 10 that contains a centered wick 12 and two bores 14 running generally alongside the wick. The candle 10 is formed of conventional candle wax, which may be scented or unscented. The top end of each bore 14 is open at the top of the candle, while the bottom end of each bore 14 is closed whereby each bore 14 forms a reservoir that can retain a fluid. A scented material 16 may be placed within one or more of the bores 14. The scented material 16 may be a liquid such as scented oil, a scented gel, or a solid material such as a stick, beads, or granules of scented wax. A plug 18, formed of wax, rubber, plastic, cork, or another suitable material, may be placed into the top of a bore 14 to close the bore 14 and retain the scented material 16 within.

The separation of the wick 12 and each bore 14 may vary depending on the diameter of the wax candle 10. The depth of penetration of the wick 12 and the bore 14 can be short of the bottom 20. Each bore 14 can contain no scented material, a single scented material, or a mixture of two or more scented materials producing a combination scent, such as apple with cinnamon to produce an apple pie scent. It should be noted that, with more than one bore 14, each bore 14 can contain a different scent thereby creating a combination scent. Also, these candles can be placed in containers of various shapes.

In FIG. 2, the wick 12 is lit to produce a flame 22. As the candle 10 burns, it melts forming a hot wax melt 24 that mixes with the fragrance material 16 in each of the bores 14 to produce a scented vapor for the enjoyment of persons present in the vicinity. When the flame 22 is extinguished, the wax melt 24 solidifies, forming a wax seal over the top of the bores 14. This wax seal will re-melt to re-open the bores 14 when the wick 12 is re-lit.

It can be appreciated that the many similar embodiments are possible, having a single bore 14 or multiple bores 14, and having a single wick 12, multiple wicks 12, or being entirely wickless. Additionally, the wax candle 10 can have external design features such as swirls, dimples, grooves, and the like.

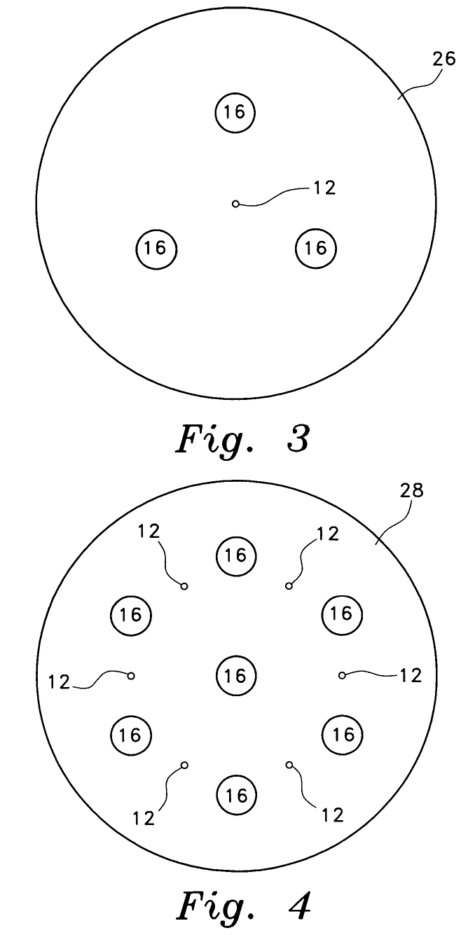

FIG. 3 is a top view of a second embodiment candle 26 having three bores or reservoirs 14 positioned around the centered wick 12.

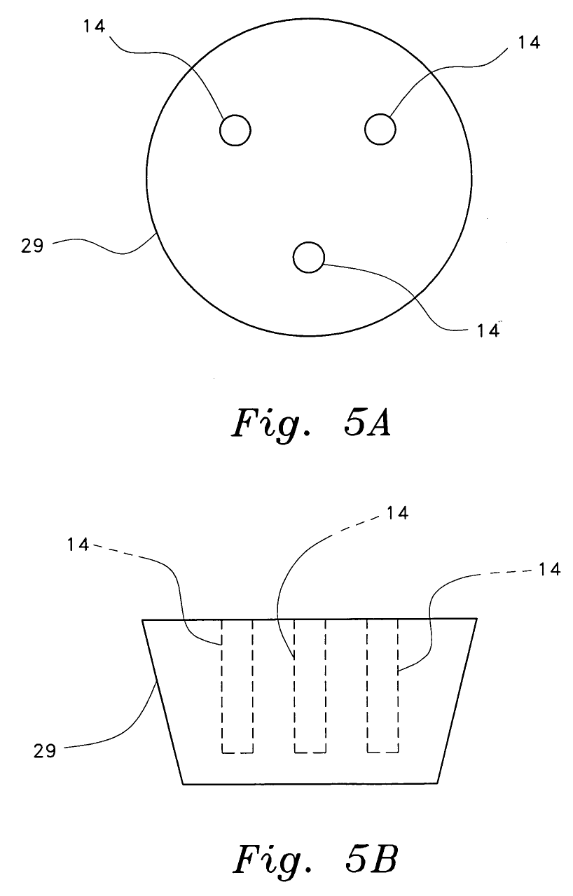

FIG. 4 is a top view of a third embodiment candle 28 having six peripheral bores 14 surrounding a centered bore 14 with six wicks 12 between the bores 14. It should be noted that the scented materials 16 contained within reservoirs 14 can be the same or different combinations of fragrances selected by the customer.

Wickless candles, known as known as “tarts”, or “melt candles”, are another form of candle used to provide a pleasing fragrance. Melt candles are typically small round, flat, wickless candles of approximately two inches in diameter and half an inch thick, although they may come in a variety of other shapes and sizes. In use, melt candles are placed into a device, typically known as a tart burner or candle warmer, where they are heated by an electrical element, a small candle, or another means to release a fragrance. In another embodiment of the present invention, illustrated in FIGS. 5A and 5B, a melt candle 29 comprises a wickless wax candle body having at least one bore 14 formed therein. A scenting material may be placed into one or more of the bores 14. The melt candle 29 of this embodiment can be used with a conventional tart burner or candle warmer.

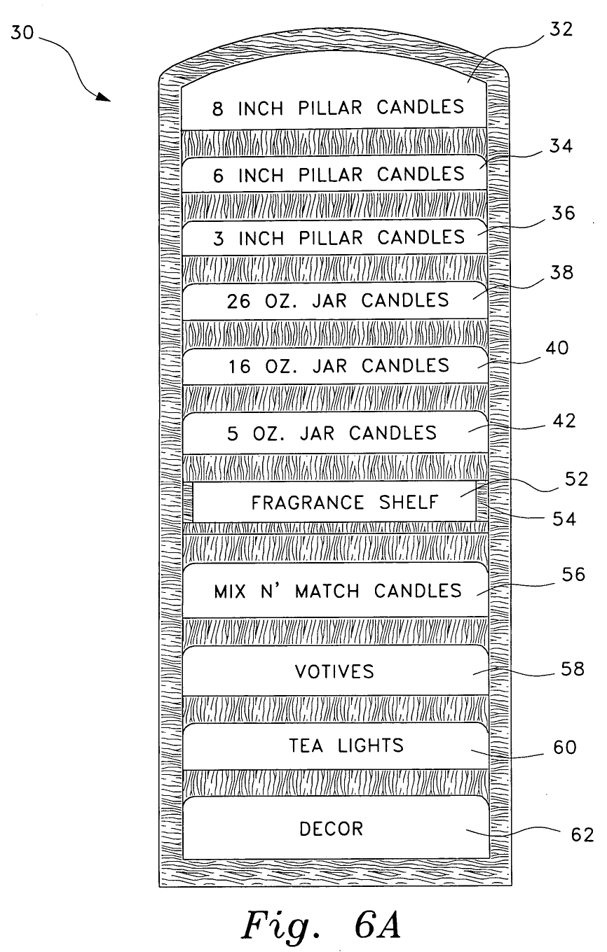

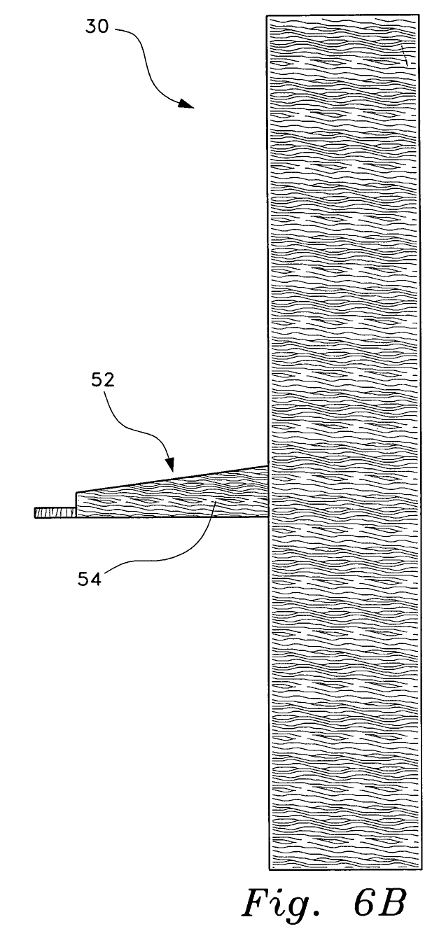

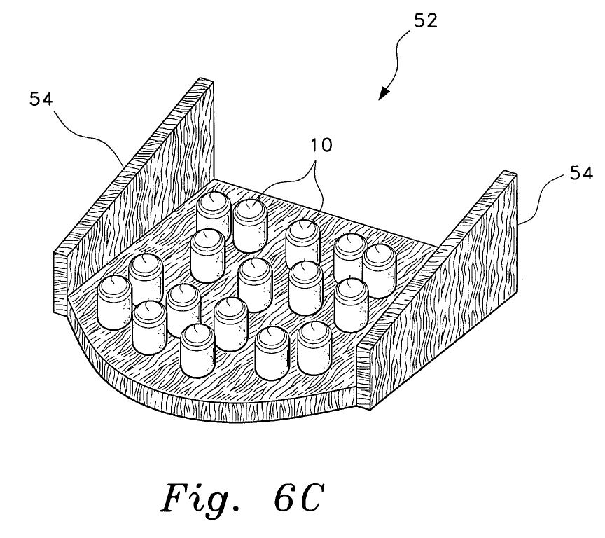

FIGS. 6A, 6B and 6C illustrate a display cabinet 30 showing various candles on each shelf. On the top shelf 8-inch tall, pillar candles 32 are displayed. On the next lower shelf, 6-inch tall pillar candles 34 are displayed. On the next lower shelf 3-inch tall, pillar candles 36 are shown. The next lower three shelves exhibit, respectfully, 26-ounce jar candles 38, 16-ounce jar candles 40, and 5-ounce jar candles 42. The next lower shelf is an extended fragrance shelf 52 wherein different fragrances are stored. The shelf 52 is extended with sides 54 as depicted best in FIG. 5B. Continuing on, “mix n′ match” candles 56 are displayed. Next, votive candles 58 are displayed above the tea light candles 60 and the decor candles 62.

FIGS. 6A-6C illustrate a method of presenting the various candles of the present invention in a display cabinet 30 having an extended shelf 52 for examining more closely the various candles such as pillar candles 32, 34 and 36 of various sizes being displayed. Some candles are contained in receptacles such as jar candles 38, 40 and 42. The candle containers can be of various designs such as tubes, vases and jars, and made of glass, ceramic, terracotta, porcelain, or metal such as brass, bronze, aluminum, steel, tin, copper, and the like. The display cabinet 30 is approximately six feet tall, and offers the assortment of fragrances in either small bottles, tubes, concentrated wax sticks, wax pellets, grains or creams, along with wax candle products.

This display cabinet 30 can hold numerous different fragrances for any of the candle options. If the consumer should desire to mix fragrances such as vanilla with nutmeg to form a customized scent, this choice is available. There will be an unlimited variety of scent materials that can be mixed for each candle. The consumer can also purchase these items and combine the scented material with the candles at his/her leisure. Thus, this display cabinet 30 can enable a retailer of candles to implement a new and different method of selling scented or unscented candles to the public.

Thus, a customer can select one or more candles from the display cabinet 30 for purchase, along with one or more scented materials to be added to the candle reservoirs. The customer may select scented materials of multiple fragrances, mixing the scented materials to form a custom scented material that is then added into the candle. The customer may mix the fragrances herself, or engage a sales person for assistance, working on the shelf 52 or another work surface to prepare the scented materials and fill the candle reservoirs.

It should be noted that the location of the different candles in the display cabinet can be varied and the choice left to the seller.

It is to be understood that the present invention is not limited to the embodiments described above, but encompasses any and all embodiments within the scope of the following claims.

1. A candle, comprising:
a wax candle body having a top and a bottom; and
at least one reservoir defined in said wax candle body, said at least one reservoir having an open top end, a closed bottom end, and a hollow interior.

2. The candle according to claim 1, wherein said at least one reservoir extends generally from the top of said wax candle body to the bottom of said wax candle body.

3. (canceled)

4. The candle according to claim 1, further comprising at least one candlewick disposed within said wax candle body.

5. The candle according to claim 4, wherein said at least one candlewick runs generally from the top of said wax candle body to the bottom of said wax candle body.

6. The candle according to claim 1, further comprising a scented material contained within at least one of said at least one reservoirs.

7. The candle according to claim 6, wherein said scented material comprises scented oil.

8. The candle according to claim 6, wherein said scented material comprises scented gel.

9. The candle according to claim 6, wherein said scented material comprises scented wax.

10-13. (canceled)

14. A method for displaying and selling candles, comprising the steps of:
displaying a plurality of candles, each of the candles comprising a wax candle body having a top and a bottom, the wax candle body having at least one reservoir defined therein, said at least one reservoir having an open top end, a closed bottom end, and a hollow interior;
displaying a plurality of scenting materials in proximity to said candles; and
offering said candles and said scenting materials for sale in various combinations according to consumer selections.

15. The method according to claim 14, wherein the step of displaying includes placing the candles are on display in a display cabinet.

16. The method according to claim 15, wherein said display cabinet is an open cabinet having a plurality of shelves.

17. The method according to claim 16, wherein said candles are placed on display on at least one of said shelves.

18. The method according to claim 16, wherein said scenting materials are placed on display on at least one of said shelves.

19. A candle kit, comprising:
a wax candle body, the wax candle body having a top and a bottom, the wax candle body having at least one reservoir defined therein, said at least one reservoir having an open top end, a closed bottom end, and a hollow interior; and
a scenting material adapted for placement into said at least one reservoir.

20. The candle kit according to claim 19, wherein said at least one reservoir extends generally from the top of said wax candle body to the bottom of said wax candle body.

21. The candle kit according to claim 19, wherein said scented material comprises a scented oil.

22. The candle kit according to claim 19, wherein said scented material comprises a scented gel.

23. The candle kit according to claim 19, wherein said scented material comprises scented wax.
